(19) United States
(12) Patent Application Publication
(10) Pub. No.: US 2011/0129548 A1
(43) Pub. Date: Jun. 2, 2011

(54) Immunopotentiating Compositions Comprising Beta-1, 3/1, 6-D-Glucan and Uses Thereof

(75) Inventors: Nino Sorgente, Savannah, GA (US); Daniel Moothart, San Clemente, CA (US)

(73) Assignee: BioAgra, LLC, Hinesville, GA (US)

(21) Appl. No.: 12/446,198

(22) PCT Filed: Oct. 19, 2007
(86) PCT No.: PCT/US07/81985

§ 371 Date: Feb. 16, 2011

Related U.S. Application Data
(60) Provisional application No. 60/853,090, filed on Oct. 20, 2006.

(51) Int. Cl. A61K 31/716; A61K 33/30; A61P 37/04; A61P 7/06; A61P 3/02

(52) U.S. Cl. 424/643; 514/54

(57) ABSTRACT

The present invention provides compositions comprising beta-1,3/1,6-D-glucan and an additive (e.g., Vitamin D, Zinc, etc.) and methods of use, e.g., enhancing the immunocompetence of animals, e.g., humans, comprising administering the compositions of the invention to the animals.

### CROSS REFERENCE TO RELATED APPLICATION

The present application claims the benefit under 35 U.S.C. §119(e) to U.S. Provisional Patent Application No. 60/853,090, which was filed Oct. 20, 2006 and is incorporated herein by reference in its entirety.

### FIELD OF INVENTION

The invention relates to compositions and methods for enhancing general health, e.g., the immunocompetence of humans and animals.

### BACKGROUND OF THE INVENTION

As a class of polysaccharides, β-(1,6) branched β-(1,3) glucans are composed of a main chain of glucose subunits linked together in (1→3) beta glycosidic linkages and branches linked to the main chain by a (1→6) beta glycosidic linkage. There are several different types of beta glucans that vary in backbone composition, branching, type of monomers or substituents resulting in polysaccharides that have different physical and biological properties.

Poly-(1-3)-β-D-glucopyranosyl-(1-6)-β-D-glucopyranose (referred to herein as “beta-1,3/1,6-D-glucan”) has been reported as a component in animal feed (U.S. Pat. Nos. 6,214,337; 7,018,986). Beta-1,3/1,6-D-glucan has also been reported to be useful in enhancing the efficacy of a fish vaccine (U.S. Pat. Nos. 5,147,862; 5,401,727; 5,189,028). Beta-1,3/1,6-D-glucan has also been shown to engage toll-like receptors, which in turn mediate macrophage activation. For example, engagement of toll-like receptors by beta-1,3/1,6-D-glucan results in the upregulation of the Vitamin D receptor and the Vitamin D-1-hydroxylase genes, which upon binding and processing of Vitamin D, lead to the induction of the antimicrobial peptide cathelicidin and the killing of intracellular Mycobacterium tuberculosis (Liu et al. (2006) Science 312:1874-5).

However, Vitamin D, specifically 1,25-dihydroxyvitamin D (1,25(OH)(2)D(3)) and its less calcemic analog 1,24(OH)(2)D(2), has been shown to inhibit the production of tumor necrosis factor alpha (TNF-α) by human peritoneal macrophages (Cohen et al. (2001) Kidney Int. 59:69-75). The inhibition of TNF-α implies an inhibitory effect on macrophages by Vitamin D.

Zinc is a critical component of many enzymes, such as the metalloproteinases, that are produced by monocytes and macrophage and regulate a number of immune functions. In macrophages, Zinc also mediates the up-regulation of metallothionines, which are responsible for lysosomal stabilization and decreased apoptosis following oxidative stress. Zinc also suppresses the production of neopterin that has been connected with depressed function of peripheral blood lymphocytes and a decrease in CD4+ T cell numbers.

### SUMMARY OF THE INVENTION

The present invention provides compositions for contributing to the health of an animal, e.g., for enhancing the immunocompetence of an animal or human, comprising therapeutically effective amounts of beta-1,3/1,6-D-glucan and an additive selected from the group consisting of Vitamin D, Zinc, and a combination of Vitamin D and Zinc. These formulations are intended to be integrated into human foodstuffs and animal feeds, to be mixed with human foodstuffs or animal feeds, or to be administered, e.g., in the form of capsules, tablets, powder, etc. to humans and to animals. Further, the invention provides methods for contributing to the health of an animal, e.g., for enhancing the immunocompetence of an animal comprising administering to the animal a composition comprising therapeutically effective amounts of beta-1,3/1,6-D-glucan and an additive selected from the group consisting of Vitamin D, Zinc, and a combination of Vitamin D and Zinc.

The primary active components of these formulations are beta-1,3/1,6-D-glucan Vitamin D and/or Zinc. In one embodiment, a composition of the invention comprises beta-1,3/1,6-D-glucan at a concentration of about 5 grams to about 500 grams per metric ton of food or feed (or about 1 mg to about 50 mg per capsule, tablet, etc.). In another embodiment, AGRASTIM® (Bioagra, LLC, Hinesville, Ga.) is used as a source of beta-1,3/1,6-D-glucan, e.g., in animal feed. In another embodiment, PURESTIM® (Bioagra, LLC, Hinesville, Ga.) is used as a source of beta-1,3/1,6-D-glucan for human consumption. In another embodiment, a composition will comprise Vitamin D at a therapeutically effective amount of about 0.5 mg to 20 g per ton of animal feed (or about 1 microgram to about 500 microgram per capsule, tablet, etc.) and zinc (e.g., as an inorganic or organic compound) in a therapeutically effective amount of about 1 mg to about 50 mg per capsule, tablet, etc., or about 1 gram to about 50 gram per ton of animal feed.

As a supplement for humans or animals, a composition may comprise about 1 mg to 50 mg of beta-1,3/1,6-D-glucan, about 1 microgram to about 500 micrograms of Vitamin D (e.g., cholecalciferol) and about 1 mg to about 50 mg of Zinc, e.g., in the form of a capsule, tablet, powder, etc. These compositions can be combined with a multivitamin or a multivitamin plus mineral formulation.

### DETAILED DESCRIPTION OF THE INVENTION

Surprisingly, poly-(1-3)-β-D-glucopyranosyl-(1-6)-β-D-glucopyranose (referred to herein as “beta-1,3/1,6-D-glucan”) and an additive (e.g., Vitamin D, Zinc, etc.) act synergistically to contribute to the general health of an animal (e.g., a mammal, e.g., a human), e.g., to increase the number of red blood cells, enhance the immunocompetence of an animal, etc. Consequently, the present invention provides compositions comprising therapeutically effective amounts of beta-1,3/1,6-D-glucan and an additive, wherein the additive is selected from the group consisting of Vitamin D, Zinc, and a combination of Vitamin D and Zinc. Further, the present invention provides methods of using the same to improve the immunocompetence of animals, e.g., mammals, e.g., humans. For example, provided herein are methods for enhancing the immunocompetence of an animal comprising administering to the animal a composition comprising poly-(1-3)-β-D-glucopyranosyl-(1-6)-β-D-glucopyranose and an additive selected from the group consisting of Vitamin D, Zinc, and Vitamin D and Zinc.

Glucans are polysaccharides consisting of glucose subunits. β-(1,6) branched β-(1,3) glucan is a naturally occurring class of glucan that can be extracted from yeast, e.g., Baker's yeast, mushrooms, plants, and some bacterial, lichen and algal species (reviewed in Chemistry and Biology of (1->3)-β-Glucans, B. A. Stone and A. E. Clarke, 1992, La Trobe University Press, Australia). For example, a yeast beta-1,3/1,6-D-glucan suitable for use in practice of the invention can be obtained from the yeast Saccharaomyces cerevisiae. Such a beta glucan may be derived from yeast cells or from a yeast cell wall preparation containing yeast beta-1,3/1-6-D-glucan; a soluble form of beta-1,3/1,6-D-glucan can be prepared from purified yeast beta-1,3/1,6-D-glucan by enzymatic degradation with a beta endoglucanase. Other beta glucans are suitable for use in practice of the invention, e.g., a beta glucan isolated from the mushroom Agaricus blazei, a beta glucan isolated from shitake mushrooms, a beta glucan derived from Sclerotium glucanicum, etc. In one embodiment of the invention, AGRASTIM® is the source of beta-1,3/1,6-D-glucan. In another embodiment, PURESTIM® is the source of beta-1,3/1,6-D-glucan.

The preferred route of administration of the beta-1,3/1,6-D-glucan as used in the invention is systemic administration, e.g., oral, subcutaneous, intramuscular, sublingual, or aerosol administration. For example, compositions comprising therapeutically effective amounts of beta-1,3/1,6-D-glucan and an additive selected from the group consisting of Vitamin D, Zinc, and a combination of Vitamin D and Zinc may be integrated into human foodstuffs or animal feed, mixed with human foodstuffs or animal feeds, or administered as supplements to humans and animals, e.g., in the form of a tablet, a capsule, powder etc., for oral ingestion.

“Therapeutically effective amounts” of beta 1,3/1,6-D-glucan, Vitamin D and/or Zinc are amounts sufficient to contribute to the general health of an animal, e.g., by enhancing the immunocompetence of the animal. Those of skill in the art will consider such factors as the animal's age, level of activity, hormone balance, general health in determining the therapeutically effective amounts, which is tailored to the subject, for example, by beginning with a low dosage and titrating the dosage to determine the effective amount. As described by the examples herein, it has been discovered that beta-1,3/1,6-D-glucan and Vitamin D may enhance the general health of an animal by, e.g., increasing the number of red blood cells. Further, it has been discovered that beta-1,3/1,6-D-glucan and Vitamin D may enhance the immunocompetence of an animal, e.g., by enhancing properties associated with an enhanced immune system (e.g., increased number of immune cells, modulated expression of cytokines, decreased expression of immunosuppressive factors, and increased effector function of immune cells, e.g., increasing nitric oxide and hydrogen peroxide production by macrophages. An ordinarily skilled artisan will recognize the different ways in which a composition of the invention may contribute to the general health of an animal, and consequently, the different methods that may be used to determine the therapeutically effective amounts for an animal.

In one embodiment of the invention, a therapeutically effective amount of beta-1,3/1,6-D-glucan is between about 5 grams to about 500 grams beta-1,3/1,6-D-glucan per metric ton of animal feed (e.g., between about 10 grams to about 100 grams beta-1,3/1,6-D-glucan per metric ton of animal feed, between about 5 grams to about 50 grams beta-1,3/1,6-D-glucan per metric ton of animal feed, between about 10 grams to about 60 grams beta-1,3/1,6-D-glucan per metric ton of animal feed, between about 80 grams to about 120 grams beta-1,3/1,6-D-glucan per metric ton of animal feed, between about 30 grams to about 80 grams beta-1,3/1,6-D-glucan per metric ton of animal feed, etc). In another embodiment, a therapeutically effect amount of beta-1,3/1,6-D-glucan is about 20 grams to about 40 grams per ton of animal feed. In other embodiments, a therapeutically effective amount of beta-1,3/1,6-D-glucan is 10, 40, 50, 60, or 100 grams per ton of animal feed. In another embodiment, a therapeutically effective amount of beta-1,3/1,6-D-glucan is between about 1 mg and about 100 mg, e.g., 5, 10, 20, 40, 50, 60, 70, 80, 90, 100 mg, etc. when beta-1,3/1,6-D-glucan is administered in the form of a capsule, a tablet, powder etc.

In one embodiment of the invention, a therapeutically effective amount of Vitamin D, e.g., cholecalciferol, is between about 0.5 mg to about 20 g per ton of animal feed. In another embodiment, a therapeutically effective amount of Vitamin D is 200 microgram, e.g., when Vitamin D is administered in the form of a capsule, a tablet, powder, etc.

In one embodiment, a therapeutically effective amount of Zinc is between about 1 g to about 50 g per ton of animal feed. In another embodiment, a therapeutically effective amount of zinc is between about 1 mg to about 50 mg, e.g., about 10 mg, when Zinc is administered in the form of a capsule, tablet, powder, etc.

All references, including patents and patent applications, cited herein are incorporated herein in their entireties.

The invention will now be described in detail by way of reference only to the following non-limiting examples.

### Example 1

### Effect of Beta-1,3/1,6-D-Glucan on Cytokines in Mice

Thirty (30) mice were fed either a control diet or a test diet containing 40 grams of beta-1,3/1,6-D-glucan (Agrastim, Bioagra LLC, Hinesville, Ga.). At day 28 the mice were bled and various cytokines in blood analyzed. The results showed that the inflammatory cytokines, interferon gamma (IFN-γ) and IL-4, respectively increased from 27 pg/ml in control diet fed mice to 41 pg/ml in test diet fed mice and from 14.3 pg/ml in control diet fed mice to 34.8 pg/ml in test diet fed mice. In contrast, the immunosuppressive cytokine, TGF-β, decreased from 1751 pg/ml in control diet fed mice to 122 pg/ml in test diet fed mice.

### Example 2

### Effect of Beta-1,3/1,6-D-Glucan on the Production of Hydrogen Peroxide by Macrophages

To determine hydrogen peroxide production, macrophage cell line RAW264 cells were plated into 96 well tissue culture plates and cultured until confluent in phenol red-free alpha-minimum essential medium, supplemented with 10% fetal bovine serum. Once confluence was reached, the media was removed, the cells were washed, and substrate and test substances were added. To insure cell number uniformity at confluence, cell numbers were determined in 8 wells; if the number of cells differed by more than 10% the plate was not used. Following an incubation period of exactly 1h the reaction was stopped and resulting fluorescence measured with an ELISA-reader at λₘₐₓ, excitation=312 nm, λₘₐₓ, emission=420 nm. Zymosan, the residue from a hot alcoholic extraction of yeast, and curdlan, a linear beta-1,3-D-glucan, were used as positive controls.

The results (Table 1) clearly show that the macrophages treated with AGRASTIM® (Bioagra LLC, Hinesville, Ga.) release a greater quantity of hydrogen peroxide.

TABLE 1
Effect of Beta-1,3/1,6-D-glucan on H₂O₂ Production by RAW264 Cells

| | Dose | Fluorescence | H₂O₂-Release |
|---|---|---|---|
| Glycans - Source | (μg/assay) | (arbitrary units) | (nmoles) |
| Control | — | 229.00 ± 4.54 | — |
| ZYMOSAN | 10 | 719.00 ± 13.21 | 2.15 |
| ZYMOSAN | 50 | 1195.00 ± 74.34 | 3.57 |
| CURDLAN | 10 | 254.00 ± 1.02 | 0.76 |
| CURDLAN | 50 | 345.00 ± 6.48 | 1.06 |
| AGRASTIM | 10 | 1185.00 ± 4.54 | 3.54 |
| AGRASTRIM | 50 | 1769.00 ± 22.07 | 5.28 |

### Example 3

### Effect of Beta-1,3/1,6-D-Glucan on the Production of Nitric Oxide by Macrophages

To determine nitric oxide production, the murine RAW264 macrophge cell line was plated in 24-well plates in DMEM supplemented with 10% FBS and antibiotics (Gentamycin [50 micrograms/ml], Amphoterecin B [5 micrograms/ml]). At confluence the media was changed to fresh media and the test substances added. Zymosan was used as the positive control.

The results (Table 2) show that Agrastim is a powerful activator of macrophages. Even though Zymosan contains approximately 40% beta-1,3/1,6-D-glucan, AGRASTIM® stimulated NO release by macrophages to a significantly greater degree compared to ZYMOSAN®.

TABLE 2
Effect of Beta-1,3/1,6-D-glucan on NO Production by RAW264 Cells

| | Dose | NO Released |
|---|---|---|
| Glycans - Source | (μg/assay) | (nmoles) |
| Control | — | — |
| ZYMOSAN | 100 | 3.9 |
| AGRASTIM | 100 | 11.6 |

### Example 4

### Effect of Vitamin D plus beta-1,3/1,6-D-glucan on Blood Cells

To determine the effect of Vitamin D plus beta-1,3/1,6-D-glucan (AGRASTIM®) on the immunocompetence of an animal, rabbits (10 per group) were given capsules of equal weight comprising 10 mg of beta-1,3/1,6-D-glucan (AGRASTIM®), 10 mg of beta-1,3/1,6-D-glucan (AGRASTIM®) plus 50 IU of Vitamin D, or 50 IU Vitamin D for 9 weeks. At the end of 9 weeks the number of white blood cells (WBCs), red blood cells (RBCs), mononuclear cells (MONs), and polymorphonuclear cells (PMNs) were determined by differential cell count. The result showed that Vitamin D unexpectedly synergized with beta 1,3/1,6-D-glucan to contribute to the general health of the animal, particularly by increasing the number of red blood cells, and enhancing properties associated with an enhanced immune system, e.g., increasing the number of white blood cells, e.g., polymophonuclear cells.

In particular, beta-1,3/1,6-D-glucan alone increased WBCs by 28% above control, and although Vitamin D alone had no effect, beta-1,3/1,6-D-glucan plus Vitamin D increased WBCs by 63% above control. Furthermore, although neither beta-1,3/1,6-D-glucan alone nor Vitamin D had significant effects on RBCs, beta-1,3/1,6-D-glucan plus Vitamin D increased the number of RBCs by 25%. Furthermore, although beta-1,3/1,6-D-glucan alone increased PMNs by 62% and Vitamin D alone increased PMNs by 29%, the combination of beta-1,3/1,6-D-glucan and Vitamin D increased PMNs by 128%. These results demonstrate that Vitamin D and beta-1,3/1,6-D-glucan unexpectedly work synergistically to contribute to the general health of an animal.

TABLE 3
Effect of Beta-1,3/1,6-D-glucan and Vitamin D on Blood cells

| | WBC | RBC | MON | PMN |
|---|---|---|---|---|
| Control* | 100 | 100 | 100 | 100 |
| Beta-G** | 128 | 103 | 211 | 162 |
| Beta-G + Vit D | 163 | 125 | 196 | 228 |
| Vitamin D | 100 | 84 | 83 | 129 |

*Data expressed as percent with the control set to 100.
**Beta-G = Beta-1,3/1,6-D-glucan

It will be understood by an ordinarily skilled artisan that these examples are for illustration purpose only and are not intended to limit the scope of the invention. Thus various modifications and addition of other minerals and vitamins can be made without departing from the spirit of the invention.

What is claimed is:

1. A composition for contributing to the health of an animal comprising therapeutically effective amounts of beta-1,3/1,6-D-glucan and an additive selected from the group consisting of Vitamin D, Zinc, and a combination of Vitamin D and Zinc.

2. The composition of claim 1, wherein the beta-1,3/1,6-D-glucan is derived from yeast.

3. The composition of claim 2, wherein the beta-1,3/1,6-D-glucan is selected from the group consisting of AGRASTIM and PURESTIM.

4. The composition of claim 1, wherein the beta-1,3/1,6-D-glucan is derived from a mushroom.

5. The composition of claim 1, wherein the beta-1,3/1,6-D-glucan is derived from a bacterium.

6. The composition of claim 1, wherein the composition further comprises animal feed, wherein the animal feed is selected from the group consisting of poultry feed, hog feed, cattle feed, horse feed, shrimp feed and pet feed.

7. The composition of claim 6, wherein the additive is Vitamin D.

8. The composition of claim 6, wherein the additive is Zinc.

9. The composition of claim 6, wherein the additive is a combination of Vitamin D and Zinc.

10. The composition of claim 9, wherein the therapeutically effective amount of beta-1,3/1,6-D-glucan is between about 10 grams to about 500 grams per ton of animal feed.

11. The composition of claim 10, wherein the therapeutically effective amount of Vitamin D is between about 0.5 mg to about 20 g per ton of animal feed.

12. The composition of claim 11, wherein the therapeutically effective amount of Zinc is between about 1 g to about 50 g per ton of animal feed.

13. A method for enhancing a property associated with an enhanced immune system in an animal comprising administering to the animal a composition comprising therapeutically effective amounts of beta-1,3/1,6-D-glucan and an additive selected from the group consisting of Vitamin D, Zinc, and a combination of Vitamin D and Zinc.

14. The method of claim 13, wherein the beta-1,3/1,6-D-glucan is derived from yeast.

15. The method of claim 14, wherein the beta-1,3/1,6-D-glucan is selected from the group consisting of AGRASTIM and PURESTIM.

16. The method of claim 13, wherein the beta-1,3/1,6-D-glucan is derived from a mushroom.

17. The method of claim 13, wherein the beta-1,3/1,6-D-glucan is derived from a bacterium.

18. The method of claim 13, wherein the composition further comprises animal feed, wherein the animal feed is selected from the group consisting of poultry feed, hog feed, cattle feed, horse feed, and pet feed.

19. The method of claim 18 wherein the additive is Vitamin D.

20. The method of claim 18, wherein the additive is Zinc.

21. The method of claim 18, wherein the additive is a combination of Vitamin D and Zinc.

22. The method of claim 21, wherein the therapeutically effective amount of beta-1,3/1,6-D-glucan is between about 5 grams to about 500 grams per ton of animal feed.

23. The method of claim 22, wherein the therapeutically effective amount of Vitamin D between about 0.5 mg to about 20 g per ton of animal feed.

24. The method of claim 23, wherein the therapeutically effective amount of Zinc is between about 1 g to about 50 g per ton of animal feed.

25. The method of claim 18, wherein the animal is a chicken, and wherein the chicken is administered a composition comprising beta-1,3/1,6-D-glucan at a therapeutically effective amount of between 10 grams and about 60 grams per ton of animal feed for the first 2 weeks of life and a composition comprising beta-1,3/1,6-D-glucan at a therapeutically effective amount of between about 5 grams and about 50 grams per ton of animal feed for the remainder of the chicken's life.

26. The method of claim 25, wherein the animal is a chicken, and wherein the chicken is administered a composition comprising beta-1,3/1,6-D-glucan at a therapeutically effective amount of 40 grams per ton of animal feed for the first 2 weeks of life and a composition comprising beta-1,3/1,6-D-glucan at a therapeutically effective amount of 20 grams per ton of animal feed for the remainder of the chicken's life.

27. The method of claim 18, wherein the animal is a turkey, and wherein the turkey is administered a composition comprising beta-1,3/1,6-D-glucan at a therapeutically effective amount of between 10 grams and about 60 grams per ton of animal feed for the first 2 weeks of life and a composition comprising beta-1,3/1,6-D-glucan at a therapeutically effective amount of between about 5 grams and about 50 grams per ton of animal feed for the remainder of the turkey's life.

28. The method of claim 27, wherein the animal is a turkey, and wherein the turkey is administered a composition comprising beta-1,3/1,6-D-glucan at a therapeutically effective amount of 40 grams per ton of animal feed for the first 6 weeks of life and a composition comprising beta-1,3/1,6-D-glucan at a therapeutically effective amount of 20 grams per ton of animal feed for the remainder of the turkey's life.

29. The method of claim 18, wherein the animal is a shrimp, and wherein the shrimp is administered a composition comprising beta-1,3/1,6-D-glucan at a therapeutically effective amount of between about 80 grams and about 120 grams per ton of animal feed.

30. The method of claim 29, wherein the animal is a shrimp, and wherein the shrimp is administered a composition comprising beta-1,3/1,6-D-glucan at a therapeutically effective amount of 100 grams per ton of animal feed.

31. The method of claim 18, wherein the animal is selected from the group consisting of a horse and a hog, and wherein the animal is administered a composition comprising beta-1,3/1,6-D-glucan at a therapeutically effective amount of between about 30 grams and about 80 grams per ton of animal feed.

32. The method of claim 31, wherein the animal is a horse, and wherein the horse is administered a composition comprising beta-1,3/1,6-D-glucan at a therapeutically effective amount of about 50 grams per ton of animal feed.

33. The method of claim 31, wherein the animal is a hog, and wherein the hog is administered a composition comprising beta-1,3/1,6-D-glucan at a therapeutically effective amount of about 60 grams per ton of animal feed.

34. The method of claim 18, wherein the animal is a pet, and wherein the pet is administered a composition comprising beta-1,3/1,6-D-glucan at a therapeutically effective amount of between about 10 grams and about 100 grams per ton of animal feed.

35. The method of claim 35, wherein the animal is a pet, and wherein the pet is administered a composition comprising beta-1,3/1,6-D-glucan at a therapeutically effective amount of about 40 grams per ton of animal feed.

36. The composition of claim 1, wherein the composition is encapsulated in a capsule and comprises between about 1 mg to about 50 mg of beta-1,3/1,6-D-glucan, between about 1 microgram to about 500 microgram of Vitamin D, and between about 1 mg to about 50 mg of Zinc

37. The composition of claim 1, wherein the composition is encapsulated in a capsule and comprises about 10 mg beta-1,3/1,6-D-glucan, about 200 microgram Vitamin D, and about 10 mg of Zinc.

38. The method of claim 1, wherein the property associated with an enhanced immune system is selected from the group consisting of an increased number of immune cells, modulated expression of cytokines, decreased expression of immunosuppressive factors, increased effector function of immune cells, and any combination of any of the above.

39. A method for increasing the number of red blood cells in an animal comprising administering to the animal a composition comprising therapeutically effective amounts of beta-1,3/1,6-D-glucan and an additive selected from the group consisting of Vitamin D, Zinc, and a combination of Vitamin D and Zinc.

40. The method of claim 39, wherein the beta-1,3/1,6-D-glucan is derived from yeast.

41. The method of claim 40, wherein the beta-1,3/1,6-D-glucan is selected from the group consisting of AGRASTIM and PURESTIM.

42. The method of claim 39, wherein the beta-1,3/1,6-D-glucan is derived from a mushroom.

43. The method of claim 39, wherein the beta-1,3/1,6-D-glucan is derived from a bacterium.

44. The method of claim 39, wherein the composition further comprises animal feed, wherein the animal feed is selected from the group consisting of poultry feed, hog feed, cattle feed, horse feed, and pet feed.

45. The method of claim 44, wherein the additive is Vitamin D.

46. The method of claim 44, wherein the additive is Zinc.

47. The method of claim 44, wherein the additive is a combination of Vitamin D and Zinc.

48. The method of claim 47, wherein the therapeutically effective amount of beta-1,3/1,6-D-glucan is between about 10 grams to about 500 grams per ton of animal feed.

49. The method of claim 48, wherein the therapeutically effective amount of Vitamin D about 0.5 mg to about 20 g per ton of animal feed.

50. The method of claim 49, wherein the therapeutically effective amount of Zinc is between about 1 g to about 50 g per ton of animal feed.

51. The method of claim 44, wherein the animal is a chicken, and wherein the chicken is administered a composition comprising beta-1,3/1,6-D-glucan at a therapeutically effective amount of between 10 grams and about 60 grams per ton of animal feed for the first 2 weeks of life and a composition comprising beta-1,3/1,6-D-glucan at a therapeutically effective amount of between about 5 grams and about 50 grams per ton of animal feed for the remainder of the chicken's life.

52. The method of claim 51, wherein the animal is a chicken, and wherein the chicken is administered a composition comprising beta-1,3/1,6-D-glucan at a therapeutically effective amount of 40 grams per ton of animal feed for the first 2 weeks of life and a composition comprising beta-1,3/1,6-D-glucan at a therapeutically effective amount of 20 grams per ton of animal feed for the remainder of the chicken's life.

53. The method of claim 44, wherein the animal is a turkey, and wherein the turkey is administered a composition comprising beta-1,3/1,6-D-glucan at a therapeutically effective amount of between 10 grams and about 60 grams per ton of animal feed for the first 2 weeks of life and a composition comprising beta-1,3/1,6-D-glucan at a therapeutically effective amount of between about 5 grams and about 50 grams per ton of animal feed for the remainder of the turkey's life.

54. The method of claim 53, wherein the animal is a turkey, and wherein the turkey is administered a composition comprising beta-1,3/1,6-D-glucan at a therapeutically effective amount of 40 grams per ton of animal feed for the first 6 weeks of life and a composition comprising beta-1,3/1,6-D-glucan at a therapeutically effective amount of 20 grams per ton of animal feed for the remainder of the turkey's life.

55. \The method of claim 44, wherein the animal is a shrimp, and wherein the shrimp is administered a composition comprising beta-1,3/1,6-D-glucan at a therapeutically effective amount of between about 80 grams and about 120 grams per ton of animal feed.

56. The method of claim 55, wherein the animal is a shrimp, and wherein the shrimp is administered a composition comprising beta-1,3/1,6-D-glucan at a therapeutically effective amount of 100 grams per ton of animal feed.

57. The method of claim 44, wherein the animal is selected from the group consisting of a horse and a hog, and wherein the animal is administered a composition comprising beta-1,3/1,6-D-glucan at a therapeutically effective amount of between about 30 grams and about 80 grams per ton of animal feed.

58. The method of claim 57, wherein the animal is a horse, and wherein the horse is administered a composition comprising beta-1,3/1,6-D-glucan at a therapeutically effective amount of about 50 grams per ton of animal feed.

59. The method of claim 57, wherein the animal is a hog, and wherein the hog is administered a composition comprising beta-1,3/1,6-D-glucan at a therapeutically effective amount of about 60 grams per ton of animal feed.

60. The method of claim 44, wherein the animal is a pet, and wherein the pet is administered a composition comprising beta-1,3/1,6-D-glucan at a therapeutically effective amount of between about 10 grams and about 100 grams per ton of animal feed.

61. The method of claim 60, wherein the animal is a pet, and wherein the pet is administered a composition comprising beta-1,3/1,6-D-glucan at a therapeutically effective amount of about 40 grams per ton of animal feed.
